(19) United States
(12) Patent Application Publication
(10) Pub. No.: US 2010/0016829 A1
(43) Pub. Date: Jan. 21, 2010

(54) APPARATUS AND METHODS FOR RETAINING A NEEDLE ON A MEDICAL INJECTOR

(76) Inventor: John F. Krumme, Woodside, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/173,084

(22) Filed: Jul. 15, 2008

(51) Int. Cl. A61M 5/34; A61M 5/20; A61M 5/31

(52) U.S. Cl. 604/506; 604/240; 604/144; 604/111

(57) ABSTRACT

Medical injectors are described herein. In some embodiments, an apparatus includes a medicament container, a needle assembly, a coupler, and a retainer. The medicament container has a distal end and a proximal end and is configured to contain a medicament. The coupler is configured to couple the needle assembly, which includes a needle and a hub, to the distal end of the medicament container. The retainer is configured to be coupled to the needle assembly and the medicament container. The retainer is configured to limit the movement of the needle assembly when the coupler fails to maintain the position of the needle assembly relative to the distal end of the medicament container.

### BACKGROUND

This invention relates generally to medical injectors and, more particularly, to apparatus and methods for retaining a needle on a medical injector.

Some known medical injectors, such as, for example, syringes, can be used to inject viscous compositions into a body of a patient. For example, some known medical injectors can be used to inject a dermal filler under the epidermis to alter the appearance of skin defects. Some known medical injectors can be used to deliver viscous fillers to a sphincter for the purpose of improving incontinence. Such medical injectors can use a small-bore needle to minimize pain and trauma to the patient. The needle can be coupled to the medical injector, for example, by threaded coupling, such as, for example, a Luer fitting.

When known medical injectors are used to inject high viscosity compositions via a small-bore needle, the pressure of the composition within the medical injector can be relatively high. In certain instances, for example, the pressure of the composition in the medical injector can be greater than 70 pounds per square inch. Accordingly, in certain circumstances, the coupler and/or the needle can fail, thereby causing the needle to become detached from the medical injector. Such needle detachment can result in discomfort for the patient, contamination of filler and/or waste of filler.

Thus, a need exists for a medical injector that is capable of retaining the needle during injection when of needle detachment and/or potential needle detachment occurs.

### SUMMARY

Medical injectors are described herein. In some embodiments, an apparatus includes a medicament container, a needle assembly, a coupler, and a retainer. The medicament container has a distal end portion and a proximal end portion, and is configured to contain a medicament. The coupler is configured to couple the needle assembly, which includes a needle and a hub, to the distal end portion of the medicament container. The retainer is configured to be coupled to the needle assembly and the medicament container. The retainer is configured to limit the movement of the needle assembly when the coupler fails to maintain the position of the needle assembly relative to the distal end portion of the medicament container.

### BRIEF DESCRIPTION OF THE DRAWINGS

FIGS. 1 and 2 are schematic illustrations of a medical injector according to an embodiment in a first configuration and a second configuration, respectively.

FIGS. 3 and 4 are perspective views of a medical injector according to an embodiment in a first configuration and a second configuration, respectively.

FIG. 5 is a side view of the medical injector illustrated in FIG. 4 in the second configuration.

FIG. 6 is a side view of the medical injector illustrated in FIG. 4 in a third configuration.

FIGS. 7 and 8 are perspective views of a medical injector according to an embodiment in a first configuration and a second configuration, respectively.

FIG. 9 is a schematic illustration of a medical injector according to an embodiment.

FIGS. 10 and 11 are perspective views of a medical injector according to an embodiment in a first configuration and a second configuration, respectively.

FIGS. 12 and 13 are front and side views, respectively, of the medical injector illustrated in FIG. 11 in the second configuration.

FIGS. 14 and 15 are perspective views of a medical injector according to an embodiment in a first configuration and a second configuration, respectively.

FIG. 16 is a perspective view of a medical injector according to an embodiment.

FIG. 17 is a flow chart of a method according to an embodiment.

### DETAILED DESCRIPTION

In some embodiments, an apparatus includes a medicament container, a needle assembly, a coupler, and a retainer. The medicament container has a distal end portion and a proximal end portion, and is configured to contain a medicament. The coupler is configured to couple the needle assembly, which includes a needle and a hub, to the distal end portion of the medicament container. The retainer is configured to be coupled to the needle assembly and the medicament container. The retainer is configured to limit the movement of the needle assembly when the coupler fails to maintain the position of the needle assembly relative to the distal end portion of the medicament container.

In some embodiments, an apparatus includes a medicament container, a needle, an adapter and a retainer. The medicament container has a distal end portion and a proximal end portion, and is configured to contain a medicament. The medicament can be, for example, a high viscosity dermal filler. The needle is coupled to the distal end portion of the medicament container. The adapter is coupled to the proximal end portion of the medicament container, and is configured to couple the medicament container to an actuator. The actuator, which can include a source of high pressure gas, is configured to move a plunger within the medicament container such that the medicament is conveyed through the needle. The retainer has a distal end portion and a proximal end portion configured to be coupled to the needle and the adapter, respectively. The retainer is configured to limit the movement of the needle with respect to the medicament container.

In some embodiments, an apparatus includes a medicament container, a needle assembly and a retainer. The medicament container is configured to contain a medicament. The needle assembly, which includes a needle and a hub, is coupled to a distal end portion of the medicament container. The retainer has a first coupling portion configured to be coupled to a portion of the medicament container and a second coupling portion defining an opening that is disposed about the hub of the needle assembly. The retainer is configured to be plastically deformed when the first coupling portion of the retainer is coupled to the portion of the medicament container.

In some embodiments, a method includes inserting a needle of a syringe into a body of a patient. The syringe is actuated such that a medicament is conveyed from a medicament container of the syringe into the body of the patient through the needle. The movement of the needle with respect to the medicament container is limited when the needle becomes decoupled from the medicament container during the actuating.

As used in this specification, the words “proximal” and “distal” refer to the direction closer to and away from, respectively, an operator (e.g., surgeon, physician, nurse, technician, etc.) who would use a medical injector during a procedure. For example, the end of a medical injector first to contact and/or be inserted into the patient's body would be the distal end, while the opposite end of the medical injector (e.g., the end of the medical injector being operated by the operator) would be the proximal end of the medical injector.

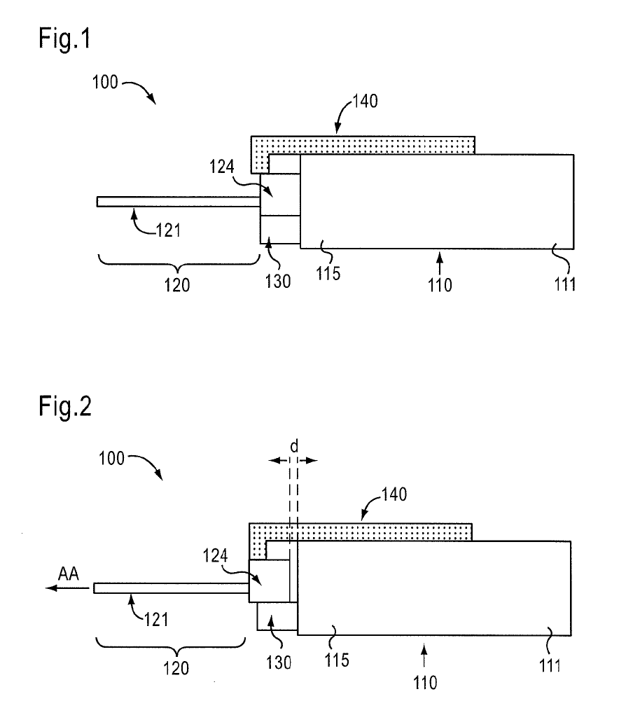

FIGS. 1 and 2 are schematic illustrations of a medical injector 100 according to an embodiment in a first configuration and a second configuration, respectively. The medical injector 100 includes a medicament container 110, a needle assembly 120, a coupler 130 and a retainer 140. The medicament container 110 has a proximal end portion 111 and a distal end portion 115. The medicament container 110 is configured to contain a medicament (not shown in FIGS. 1 and 2), such as, for example, a dermal filler, a sub-dermal filler, a therapeutic substance for mesotherapy or the like. The medicament container 110 can include a plunger (not shown in FIGS. 1 and 2) configured to displace the medicament within the medicament container 110.

The needle assembly 120 includes a needle 121 and a hub 124. The needle can be, for example, a small-bore needle (e.g., a needle having a bore size less than or equal to that of a 27 gauge needle) configured to convey the medicament from the medicament container 110 to the patient's body. The needle 121 and the hub 124 are collectively coupled to the distal end portion 115 of the medicament container 110 via the coupler 130 such that the needle 121 is in fluid communication with the medicament container 110. The coupler 130 couples the needle assembly 120 and the medicament container 110 such that the medicament does not leak from the interface between the medicament container 110 and the needle assembly 120 when the medicament is conveyed from the medicament container 110 via the needle 121. Said another way, the coupler 130 provides a substantially fluid-tight seal (i.e., a seal that that substantially prevents a liquid and/or a gas from passing therethrough) between the needle assembly 120 and the medicament container 110. In some embodiments, the fluid-tight seal can be a hermetic seal (i.e., a seal that substantially prevents a gas from passing therethrough). Moreover, the coupler 130 is configured to maintain a position of the needle assembly 120 relative to the distal end 115 of the medicament container 110.

In some embodiments, the needle assembly 120 can be removeably coupled to the medicament container 110 by the coupler 130. Said another way, in some embodiments, the needle assembly 120 can be attached to the medical injector 100 prior to an injection or removed from the medical injector 100 after an injection. The coupler 130 can be, for example, a press-fit Luer fitting (e.g., a Luer-Slip™ fitting), a twist-on Luer fitting (e.g., a Luer-Lok™ fitting) and/or the like.

The retainer 140 is coupled to the needle assembly 120 and the medicament container 110. The retainer 140, which can be, for example, a plastic strip (e.g., a Mylar® strip), is configured to limit movement of the needle assembly 120 relative to the distal end portion 115 of the medicament container 110 when the coupler 130 and/or a portion of the needle assembly 120 fails. Said another way, the retainer 140 is configured to retain the needle assembly 120 relative to the medicament container 110 when the coupler 130 and/or a portion of the needle assembly 120 fails. Although the retainer 140 is shown as being coupled to the hub 124 of the needle assembly 120, in some embodiments, the retainer 140 can be coupled to the needle 121 of the needle assembly 120. In other embodiments, the retainer 140 can be coupled to both the needle 121 and the hub 124 of the needle assembly 120.

In use, a distal portion of the needle 121 can be inserted into the patient's body (not shown in FIGS. 1 and 2). The medical injector 100 can then be actuated such that the medicament in the medicament container 110 is conveyed from the medicament container 110 to the patient's body via the needle 121. As shown in FIG. 2, the medicament moves from the needle 121 in a direction AA. In some instances, the pressure of the medicament in the medicament container 110 can cause the coupler 130 and/or a portion of the needle assembly 120 to fail such that the needle assembly 120 detaches from the medicament container 110 and moves, for example, a distance d substantially in the direction AA, as shown in FIG. 2. Said another way, in some instances, the coupler 130 can fail to maintain the position of the needle assembly 120 relative to the medicament container 110 during an injection event. When such detachment occurs, the retainer 140 limits the movement of the needle assembly 120 relative to the medicament container 110, for example, in the direction AA. More particularly, the retainer 140 prevents the needle assembly 120 from moving relative to the medicament container 110 greater than the distance d. Although the needle assembly 120 is shown as moving relative to the medicament container 110 a distance d during a needle detachment event, in some embodiments, the retainer 140 is configured to maintain a position of the needle assembly 120 such that the needle assembly 120 does not move relative to the medicament container 110 when the coupler 130 fails. Said another way, in some embodiments, the retainer 140 is configured to maintain a position of the needle assembly 120 thereby preventing a needle detachment event.

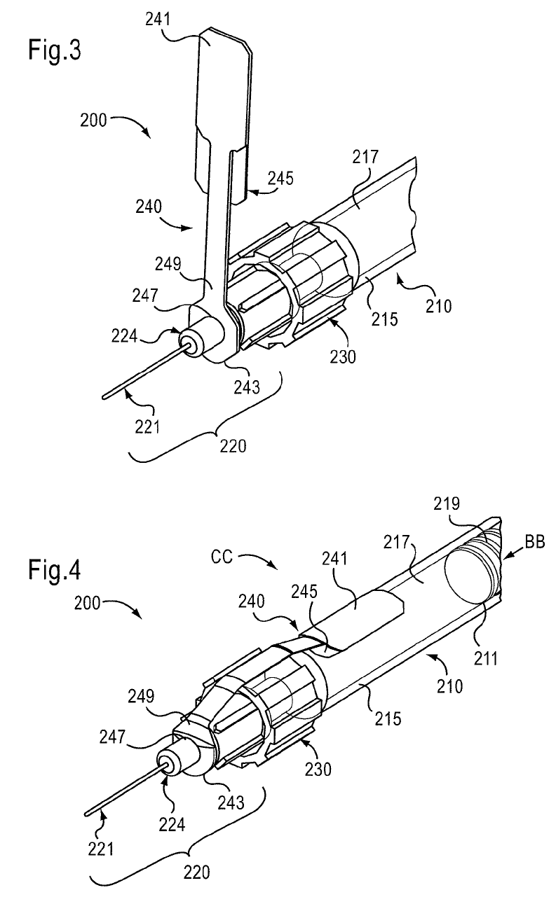

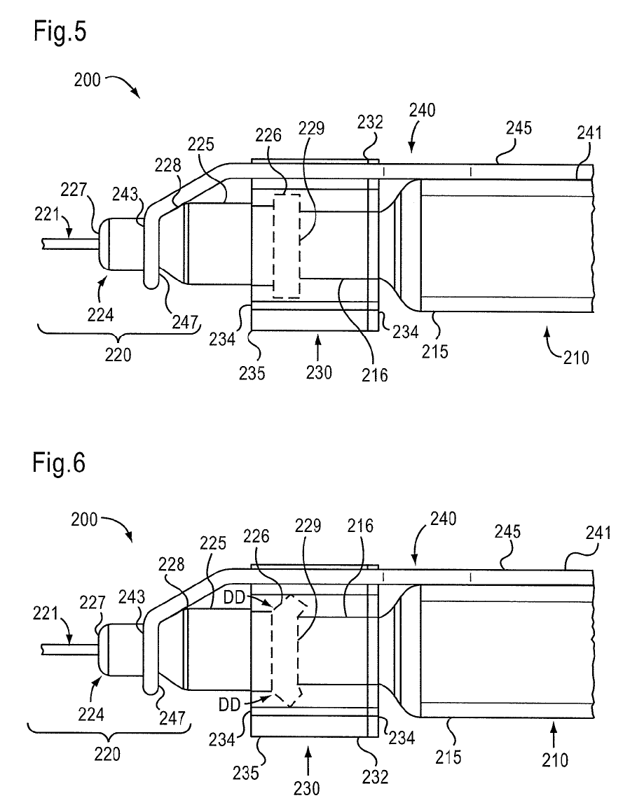

FIGS. 3 and 4 are perspective views of a medical injector 200 according to an embodiment in a first configuration and a second configuration, respectively. FIGS. 5 and 6 are side views of the medical injector 200 in the second configuration and a third configuration, respectively. The medical injector 200 includes a medicament container 210, a needle assembly 220, a coupler 230 and a retainer 240. The medicament container 210, which can be constructed of glass, plastic, or the like, is configured to contain a medicament 217, such as, a dermal filler, a sub-dermal filler, a therapeutic substance for mesotherapy or the like. The medicament container 210 has a proximal end portion 211 and a distal end portion 215. The distal end portion 215 of the medicament container 210, as shown in FIGS. 5 and 6, includes a protrusion 216 that is tapered, for example, at a six degree angle. The protrusion 216 includes a lip (not shown). As described in more detail herein, the tapered protrusion 216 is configured to be matingly disposed within a hub 224 of the needle assembly 220 to provide a substantially fluid-tight seal. The medicament container 210 includes a plunger 219 (see e.g., FIG. 4) configured to displace the medicament 217 within the medicament container 210. More particularly, when the medical injector 200 is actuated, the plunger 219 is moved within the medicament container 210, in a direction indicated by the arrow BB in FIG. 4, causing the medicament 217 to be conveyed into a patient's body.

The needle assembly 220 includes a needle 221 and a hub 224. The needle 221 can be, for example, a small-bore needle (e.g., a needle having a bore size less than or equal to that of a 27 gauge needle) configured to convey the medicament 217 from the medicament container 210 to the patient's body. As shown in FIGS. 5 and 6, the hub 224 includes a distal end portion 227 and a proximal end portion 225. The distal end portion 227 of the hub 224 defines a shoulder 228 against which a portion of the retainer 240 is disposed, as described herein. The needle 221 is disposed within the distal end portion 227 of the hub 224. The proximal end portion 225 of the hub 224 includes a flanged portion 226 and an opening 229. The opening 229 of the hub 224 receives the tapered protrusion 216 of the medicament container 210 to produce a substantially fluid-tight seal between the medicament container 210 and the needle assembly 220. The flanged portion 226 of the hub 224 is threadedly coupled to the coupler 230, as described herein. In this manner, the needle assembly 220 is coupled to the distal end portion 215 of the medicament container 210 via the coupler 230 such that the needle 221 is in fluid communication with the medicament container 210.

The coupler 230 includes a distal end portion 235, a proximal end portion 232, and defines an opening 234 therethrough. A portion of the opening 234 includes female threads (not shown) configured to receive the flanged portion 226 of the hub 224 to threadedly couple the hub 224 to the coupler 230. The coupler 230 is disposed about the protrusion 216 such that the protrusion 216 is within the opening 234 of the coupler 230. More specifically, a portion of the proximal end portion 232 of the coupler 230 engages the lip (not shown) of the protrusion 216 such that the coupler 230 cannot move axially, relative to the medicament container 210. Similarly stated, the proximal end portion 232 and the lip of the protrusion 216 form a snap-fit that allows the coupler 230 to rotate relative to the medicament container 210 and needle assembly 220, but limits axial movement of the coupler 230 relative to the medicament container 210. As shown in FIGS. 5 and 6, the hub 224 is disposed about the protrusion 216 such that a portion of the protrusion 216 is within the opening 229 of the hub 224. In this manner, the coupler 230, the hub 224 and the protrusion 216 collectively form a substantially fluid-tight seal. Said another way, the coupler 230 is configured to maintain a position of the needle assembly 220 relative to the distal end portion 215 of the medicament container 210. The coupler 230 can be, for example, a press-fit Luer fitting, a twist-on Luer-lock fitting and/or the like.

The retainer 240 is constructed from any suitable flexible material, such as, for example, Mylar®. The retainer 240 includes a proximal end portion 241 and a distal end portion 243. The distal end portion 243 of the retainer 240 defines an opening 247 within which the hub 224 of the needle assembly 220 is disposed such that a portion of the distal end portion 243 of the retainer 240 is in contact with the shoulder 228 of the hub 224. In some embodiments, the opening 247 of the retainer 240 and the shoulder 228 of the hub 224 collectively form an interference fit such that the distal end portion 243 of the retainer 240 is secured to the needle assembly 220.

The proximal end portion 241 of the retainer 240 includes an adhesive portion 245. The adhesive portion 245 can include, for example, a pressure sensitive adhesive having adhesive properties that are enhanced when pressure is applied. The adhesive portion 245 adhesively couples the proximal end portion 241 of the retainer 240 to the outer surface of the medicament container 210. A portion of the proximal end portion 241 of the retainer 240 can be devoid of adhesive and, therefore, can be used as a pull tab configured to decouple the retainer 240 from the medicament container 210. For example, upon completion of an injection, a doctor can remove the retainer 240 by exerting a force on the pull tab such that the adhesive portion 245 of the retainer 240 peels off of the medicament container 210. In this manner, the retainer 240 is removeably coupled to the medicament container 210.

In use, the retainer 240 is coupled to the medical injector 200 before an injection occurs. First, the opening 247 defined by the distal end portion 243 of the retainer 240 is placed about the hub 224 of the needle assembly 220 such that the distal end portion 243 of the retainer 240 engages the shoulder 228 of the hub 224, limiting any further movement. The flexible retainer 240 is bent at location 249 such that the proximal end portion 241 is disposed against the outer surface of the medicament container 210, as indicated by arrow CC in FIG. 4. The proximal end portion 241 of the retainer 240 is coupled to the outer surface of the medicament container 210 by applying a pressure to the adhesive portion 245 of the retainer 240. Such pressure can be applied, for example, by the user pressing the user's thumb on the adhesive portion 245 of the retainer 240. In this manner, the user can couple the retainer 240 to the needle assembly 220 and the medicament container 210 after the needle assembly 220 is in place. Similarly stated, this arrangement allows the user to couple the retainer 240 to the medical injector 200 without breaking the seal between the needle assembly 240 and the medicament container 210. Said another way, this arrangement allows the retainer 240 to be coupled to the medical injector 200 without compromising the sterility of the needle assembly 220 and/or the medicament container 210.

A distal end portion of the needle 221 of the medical injector 200 can be inserted into the patient's body (not shown). The medical injector 200 can then be actuated such that the medicament 217 in the medicament container 210 is conveyed from the medicament container 210 to the patient's body via the needle 221. As shown in FIG. 4, when the medicament container 210 is actuated, the plunger 219 moves the medicament 217 through the needle 221 in a direction BB. As described above, in some instances, the pressure of the medicament 217 in the medicament container 210, which can exceed 70 p.s.i., can cause the coupler 230 and/or a portion of the needle assembly 220 to fail such that the needle assembly 220 detaches from the medicament container 210. For example, as shown in FIG. 6, the pressure of the medicament 217 can cause a portion of the flanged portion 226 of the hub 224 to deform, as shown by arrows DD. Such deformation can cause the flanged portion 226 of the hub 224 to decouple from the female threads of the coupler 230. Said another way, the coupler 230 fails to limit movement of the needle assembly 220 when the flanged portion 226 of the hub 224 deforms, resulting in needle detachment or a potential needle detachment. When such a failure occurs, however, the retainer 240 holds the needle assembly 220 securely to the medicament container 210 such that needle detachment avoided. More particularly, the retainer 240 prevents the needle assembly 220 from moving relative to the medicament container 210.

Although the retainer 240 is shown and described above as being disposed about the needle 221 after the needle assembly 220 is coupled to the medicament container 210, in other embodiments, the retainer 240 can be disposed about the needle 221 before the needle assembly 220 is coupled to the medicament container 210. For example, in some embodiments, the retainer 240 can be disposed about the needle 221 as a part of manufacturing and/or assembly of the needle assembly 220. Moreover, the needle 221 and the distal end portion 243 of the retainer 240 can be covered by a protective cover (not shown in FIGS. 3-6) after the retainer 240 is disposed about the needle 221. In this manner, the distal end portion 243 of the retainer 240 can be coupled to needle assembly 220 without compromising the sterility of the needle assembly 220.

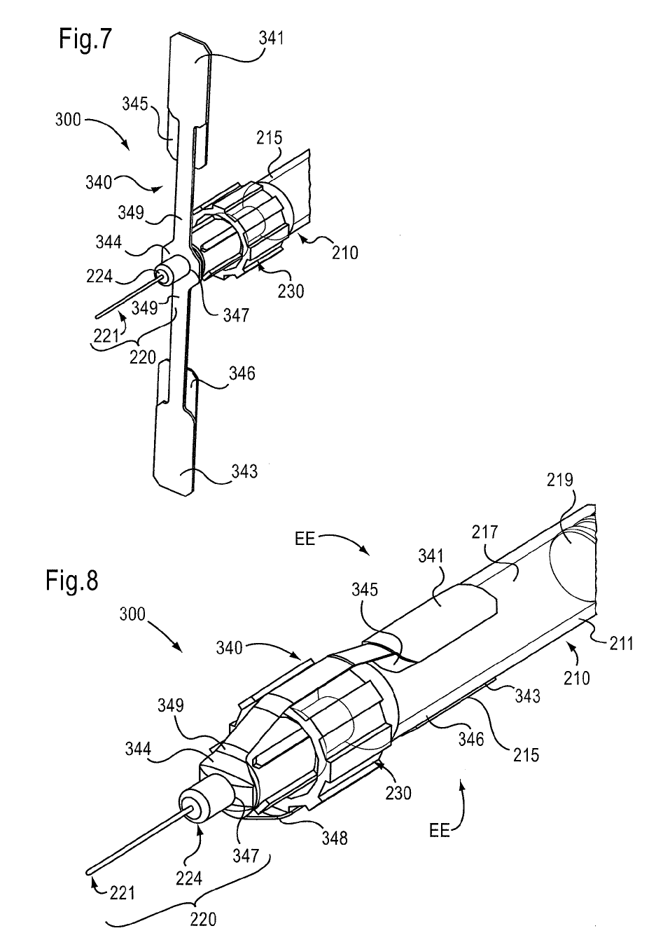

FIGS. 7 and 8 are perspective views of a medical injector 300 according to an embodiment having a retainer 340 in a first configuration and a second configuration, respectively. The medical injector 300 includes a medicament container 210, a needle assembly 220, a coupler 230 and a retainer 340. The structure and operation of the medicament container 210, the needle assembly 220 and the coupler 230 are discussed above. Accordingly, only the retainer 340 is described in detail below. The retainer 340 includes a first end portion 341, a second end portion 343 and a central portion 344. The central portion 344 of the retainer 340 defines an opening 347 within which the hub 224 of the needle assembly 220 can be disposed such that the central portion 344 of the retainer 340 abuts the shoulder 228 of the hub 224. The retainer 340 is constructed from a flexible material, such as, for example, Mylar®.

The first end portion 341 of the retainer 340 includes a first adhesive portion 345 and the second end portion 343 of the retainer 340 includes a second adhesive portion 346. As described above, the adhesive portions 345 and 346 can include any pressure sensitive adhesive. Each of the adhesive portions 345 and 346 are configured similar to the adhesive portion 245 described above such that the adhesive portions 345 and 346 can adhesively couple the retainer 340 to the outer surface of the medicament container 210. Additionally, each of the end portions 341 and 343 of the retainer 340 can include a portion devoid of adhesive, which can be used as a pull tab configured to decouple the retainer 340 from the medicament container 210. Similar to the previous example, upon completion of an injection, a doctor can remove the retainer 340 by exerting a force on each of the pull tabs, though not necessarily simultaneously, such that the adhesive portions 345 and 346 of the retainer 340 peel off of the medicament container 210. In this manner, the retainer 340 is removeably coupled the medicament container 210.

In use, the retainer 340 is coupled to the medical injector 300 before an injection occurs. First, the opening 347 defined by the central portion 344 of the retainer 340 is placed about the hub 224 of the needle assembly 220 such that the central portion 344 of the retainer 340 engages the shoulder 228 of the hub 224, limiting any further movement. The end portions 341 and 343 are then disposed against the outer surface of the medicament container 210, as indicated by arrows EE in FIG. 8. The end portions 341 and 343 of the retainer 340 are coupled to the outer surface of the medicament container 210 by applying a pressure to the adhesive portions 345 and 346 of the retainer 340, as described above. Upon coupling the retainer 340 to the medicament container 210, the medical injector 300 functions similar to medical injector 200, as described above.

Although the retainer 340 is shown and described above as being disposed about the needle 321 after the needle assembly 320 is coupled to the medicament container 310, in other embodiments, the retainer 340 can be disposed about the needle 321 before the needle assembly 320 is coupled to the medicament container 310. For example, in some embodiments, the retainer 340 can be disposed about the needle 321 as a part of manufacturing and/or assembly of the needle assembly 320, as described above.

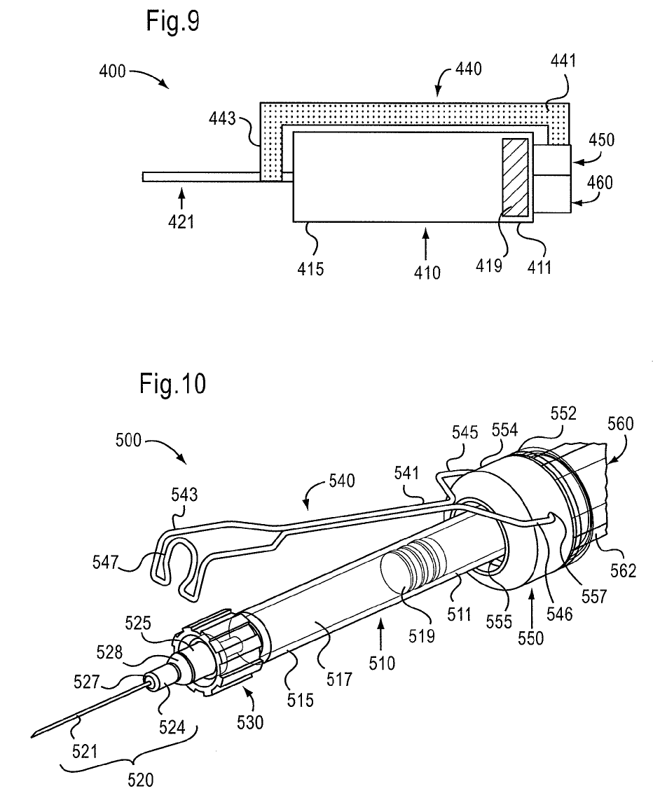

FIG. 9 is a schematic illustration of a medical injector 400 according to yet another embodiment. The medical injector 400 includes a medicament container 410, a needle 421, a retainer 440, an adapter 450 and an actuator 460. The medicament container 410 has a proximal end portion 411 and a distal end portion 415. The medicament container 410, which can be constructed of glass, for example, is configured to contain a medicament (not shown), such as, for example, a dermal filler, a sub-dermal filler, a therapeutic substance for mesotherapy or the like. The medicament container 410 includes a plunger 419 configured to displace the medicament within the medicament container 410.

The needle 421 is coupled to the distal end portion 415 of the medicament container 410 such that the needle 421 is in fluid communication with the medicament container 410. The needle 421 can be, for example, a small-bore needle (e.g., a needle having a bore size less than or equal to that of a 27 gauge needle) configured to convey the medicament from the medicament container 410 to the patient's body. In some embodiments, the needle 421 can be coupled to a hub (not shown in FIG. 9), which is coupled to the distal end portion 415 of the medicament container 410 via a coupler, as described above.

The actuator 460 is operatively coupled to the proximal end portion 411 of the medicament container 410 via the adapter 450. In some embodiments, the adapter 450 can removeably couple the actuator 460 to the medicament container 410. For example, in some embodiments, the adapter 450 can threadedly couple the actuator 460 to the medicament container 410. In other embodiments, the adapter 450 can form a press-fit coupling between the actuator and the medicament container.

The actuator 460 is configured to move the plunger 419 within the medicament container 410 such that the medicament is conveyed to the patient's body via the needle 421. In some embodiments, the actuator 460 can include a source of pressurized fluid that exerts a force on the plunger 419. In some such embodiments, the actuator 460 can also include a pressure amplifier, configured to receive a fluid at a first pressure and to produce a force on the plunger 419 such that a pressure of the medicament within the medicament container 410 is at a second pressure greater than the first pressure. In other embodiments, the actuator 460 can be a mechanical device configured to move the plunger 419. For example, in some embodiments, the actuator can include a stepper motor configured to move the plunger 419 within the medicament container 410.

The retainer 440 includes a proximal end portion 441 and a distal end portion 443. The proximal end portion 441 of the retainer 440 is coupled to the adapter 450. The distal end portion 443 of the retainer 440 is coupled to the needle 421. The retainer 440, which can be, for example, a rigid member, is configured to limit movement of the needle 421 relative to the medicament container 410, when the distal end portion 443 of the retainer 440 is coupled to the needle 421 and the proximal end portion 441 of the retainer 440 is coupled to the adapter 450. Moreover, the retainer 440 is configured to limit the movement of the medicament container 410 relative to the adapter 450, when the distal end portion 443 of the retainer 440 is coupled to the needle 421 and the proximal end portion 441 of the retainer 440 is coupled to the adapter 450. Said another way, the retainer 440 is configured to retain the needle 421 and/or the medicament container 410 relative to the adapter 450 and/or the actuator 460.

In use, a distal end portion of the needle 421 can be inserted into the patient's body (not shown in FIG. 9). The medical injector 400 can then be actuated such that the actuator 460 moves the plunger 419 within the medicament container 410. Movement of the plunger 419 causes the medicament in the medicament container 410 to be conveyed from the medicament container 410 to the patient's body via the needle 421. In some instances, the pressure of the medicament in the medicament container 410, which can exceed 70 p.s.i., can cause the needle 421 and/or the medicament container 410 to fail such that the needle 421 detaches from the medicament container 410 and/or the medicament container 410 detaches from the adapter 450. When such detachment occurs, the retainer 440 limits the movement of the needle 421 relative to the medicament container 410

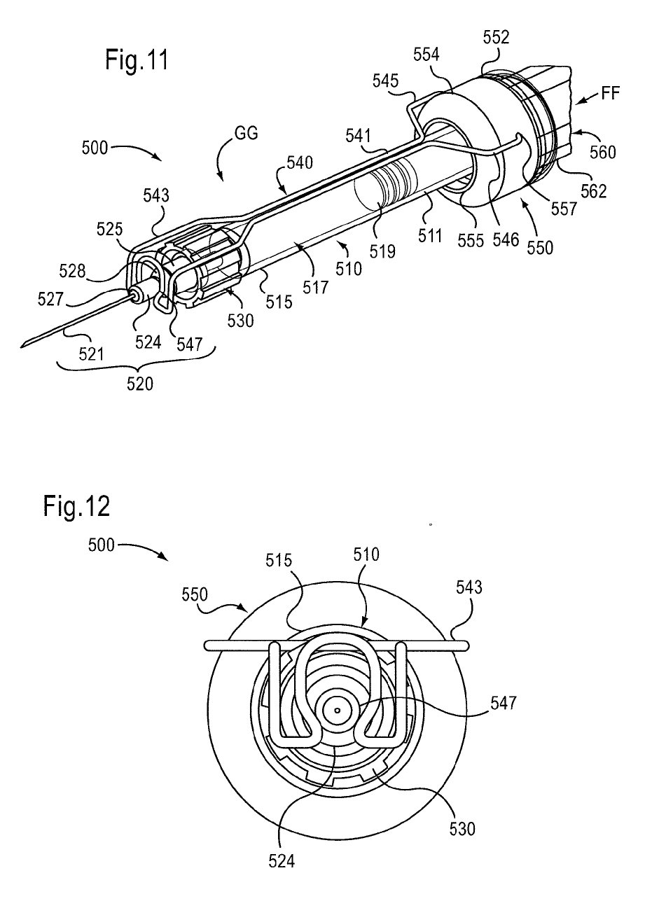

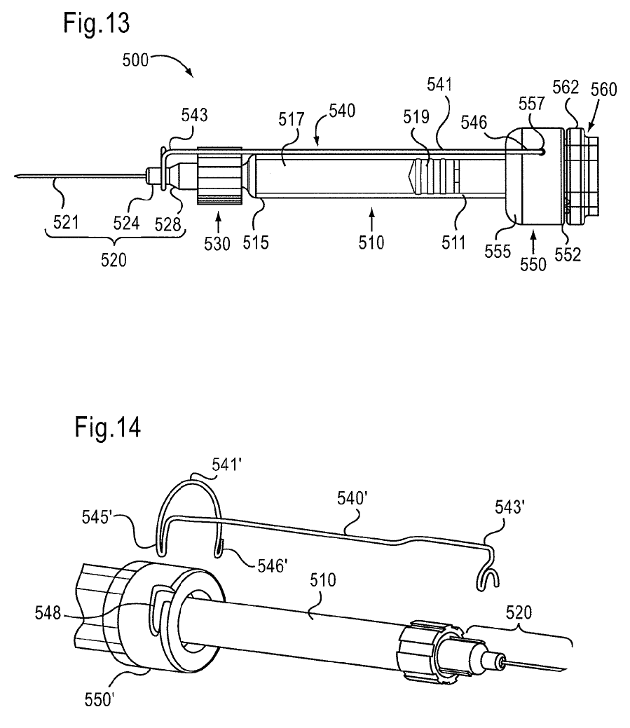

FIGS. 10 and 11 are perspective views of a medical injector 500 according to an embodiment in a first configuration and a second configuration, respectively. FIGS. 12 and 13 are front and side views, respectively, of the medical injector 500 in the second configuration. The medical injector 500 includes a medicament container 510, a needle assembly 520, a coupler 530, a retainer 540, an adapter 550 and an actuator 560. The medicament container 510 has a proximal end portion 511 and a distal end portion 515. The medicament container 510, which can be constructed of glass, for example, is configured to contain a medicament 517, such as, for example, a dermal filler, a sub-dermal filler, a therapeutic substance for mesotherapy or the like. The medicament container 510 includes a plunger 519 configured to displace the medicament 517 within the medicament container 510. More particularly, when the medical injector 500 is actuated, the plunger 519 is moved within the medicament container 510, in a direction indicated by the arrow FF in FIG. 11, causing the medicament 517 to be conveyed into a patient's body.

The needle assembly 520 includes a needle 521 and a hub 524. The needle 521 can be, for example, a small-bore needle (e.g., a needle having a bore size less than or equal to that of a 27 gauge needle) configured to convey the medicament 517 from the medicament container 510 to the patient's body. As shown in FIGS. 10 and 11, the hub 524 includes a distal end portion 527 and a proximal end portion 525. The distal end portion 527 of the hub 524 defines a shoulder 528 against which a portion of the retainer 540 is disposed. The needle 521 is disposed within the distal end portion 527 of the hub 524. The needle 521 and the hub 524 are collectively coupled to the distal end portion 515 of the medicament container 510 via the coupler 530 such that the needle 521 is in fluid communication with the medicament container 510. The coupler 530 provides a substantially fluid-tight seal between the needle assembly 520 and the medicament container 510 such that the medicament 517 does not leak from the interface between the medicament container 510 and the needle assembly 520 when the medicament 517 is conveyed from the medicament container 510 via the needle 521, as described above.

The adapter 550 includes a proximal end portion 552 and a distal end portion 555, and defines an opening (not shown) therethrough. A portion of the opening includes female threads (not shown) configured to receive male threads (not shown) of the actuator 560 to threadedly couple the actuator 560 to the adapter 550. Similarly stated, the proximal end portion 552 of the adapter 550 can be removeably coupled to the actuator 560. The adapter 550 is disposed about a flanged portion (not shown) of the proximal end portion 511 of the medicament container 510, as described above, such that the distal end portion 555 of the adapter 550 is coupled to the proximal end portion 511 of the medicament container 510. In this manner, the medicament container 510 and the actuator 560 are removeably and/or operatively coupled to the adapter 550. Additionally, the outer surface of the adapter 550 defines a first opening 554 and a second opening 557. The first opening 554 and the second opening 557 of the adapter 550 are configured to receive and retain a portion of the retainer 540, as discussed herein.

The actuator 560 is configured to move the plunger 519 within the medicament container 510 such that the medicament 517 is conveyed to the patient's body via the needle 521. In some embodiments, the actuator 560 can include a source of pressurized fluid (i.e., a gas or a liquid) that exerts a force on the plunger 519. In some embodiments, for example, the actuator can include a source of pressurized fluid having a pressure of at least 70 p.s.i. In other embodiments, the actuator can include a source of pressurized fluid having a pressure of at least 100 p.s.i. In yet other embodiments, the actuator can include a source of pressurized fluid having a pressure of at least 150 p.s.i.

In some such embodiments, the actuator 560 can also include a pressure amplifier, configured to receive a fluid at a first pressure and to produce a force on the plunger 519 such that a pressure of the medicament within the medicament container 510 is at a second pressure greater than the first pressure. For example, in some embodiments, the actuator 560 can be configure to receive a fluid at a pressure of approximately 76 p.s.i. and produce a force on the plunger 519 such that a pressure of the medicament within the medicament container 510 is approximately 250 p.s.i. In other embodiments, the actuator 560 can actuate the plunger 519 indirectly by acting on a rod (not shown) coupled to the plunger 519 such that the rod moves the plunger 519. For example, in some such embodiments, the actuator 560 can be a mechanical device (e.g., a stepper motor) configured to move the plunger.

The retainer 540, which can be constructed from any suitable rigid material, includes a proximal end portion 541 and a distal end portion 543. The proximal end portion 541 of the retainer 540 is coupled to the adapter 550. The proximal end portion 541 includes a first coupling portion 545 and a second coupling portion 546. The first coupling portion 545 fits into the first opening 554 defined by the adapter 550. Similarly, the second coupling portion 546 fits into the second opening 557 defined by the adapter 550. More particularly, the distance between the coupling portions 545 and 546 is smaller than the distance between the openings 554 and 557 such that the coupling portions 545 and 546 snap into the openings 554 and 557 to form a secure fit. In this manner, the coupling portions 545 and 546 can rotate within the openings 554 and 557 such that the retainer 540 can move relative to the medicament container 510 between a first position and a second position when the coupling portions 545 and 546 are installed, as shown in FIGS. 10 and 11, respectively. The coupling portions 545 and 546, however, engage the openings 554 and 557 such that axial movement of the retainer 540 relative to the adapter 550 and/or the actuator 560 is limited.

The distal end portion 543 of the retainer 540 defines an opening 547 configured to be disposed about the hub 524 of the needle assembly 520. As shown in FIG. 12, the opening 547 of the retainer 540 and the hub 524 form an interference fit such that the distal end portion 543 of the retainer 540 is secured to the needle assembly 520. More particularly, a portion of the opening 547 of the retainer 540 has a size smaller than the diameter of the hub 524 such that the opening 547 of the retainer 540 snaps into the hub 524 to form a secure fit.

In use, the retainer 540 is coupled to the medical injector 500 before an injection occurs. First, the first coupling portion 545 and second coupling portion 546 of the retainer 540 are disposed within the openings 554 and 557 of the adapter 550, respectively. The retainer 540 is then moved from the first position to the second position such that the distal end portion 543 of the retainer 540 is snapped over the hub 524 of the needle assembly 520, as indicated by arrow GG in FIG. 11.

A distal end portion of the needle 521 of the medical injector 500 can be inserted into the patient's body (not shown). The medical injector 500 can then be actuated such that the medicament 517 in the medicament container 510 is conveyed from the medicament container 510 to the patient's body via the needle 521. More specifically, the actuator 560 causes the plunger 519 to move the medicament 517 from the needle 521 in a direction FF, as shown in FIG. 11. In some instances, the pressure of the medicament 517 in the medicament container 510, which can exceed 70 p.s.i., can cause the coupler 530 and/or a portion of the needle assembly 520 to fail such that the needle assembly 520 detaches from the medicament container 510. In other instances, the pressure of the medicament 517 in the medicament container 510 can cause the medicament container 510 and/or the adapter 550 to fail such that the medicament container 510 detaches from the adapter 550. When such detachment occurs, the retainer 540 substantially maintains the position of the needle assembly 520 and/or the medicament container 510. Said another way, the retainer 540 prevents the needle assembly 520 and/or the medicament container 510 from moving relative to the adapter 550 when the medicament container 510 and/or the adapter 550 fail.

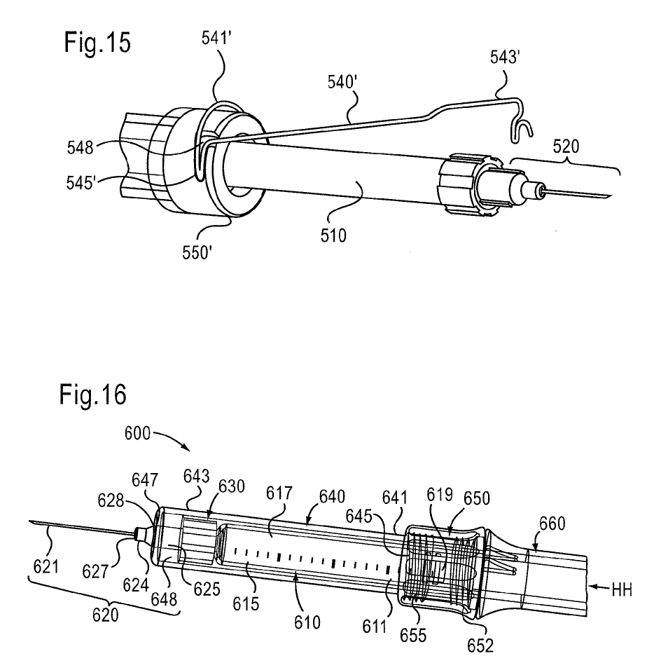

Although the adapter 550 is shown as defining openings 554 and 557 within which the coupling portions 545 and 546 can be disposed to couple the retainer 540 to the adapter 550, in other embodiments, the retainer 540 can be coupled to the adapter 550 by any suitable mechanism. In some embodiments, for example, the retainer 540 can be coupled to the adapter 550 by a ball-and-socket joint. In other embodiments, the retainer 540 can be disposed within a groove of the adapter 550 to couple the retainer 540 to the adapter 550. For example, FIGS. 14 and 15 show a medical injector 500′ having an adapter 550′ that defines grooves 548. The coupling portions 545′ and 546′ of the retainer 540′ are configured to be disposed within the grooves 548 to couple the retainer 540′ to the adapter 550′. As shown in FIG. 15, the proximal end portion 541′ of the retainer 540′ is spaced apart from the outer surface of the adapter 550′ when the coupling portions 545′ and 546′ of the retainer 540′ are disposed within the grooves 548. In this manner, the retainer 540′ can rotate relative to the adapter 550′ and/or the medicament container 510 to couple the distal end portion 543′ of the retainer to the needle assembly 520.

FIG. 16 is a perspective view of a medical injector 600 according to another embodiment. The medical injector 600 includes a medicament container 610, a needle assembly 620, a coupler 630, a retainer 640, an adapter 650, and an actuator 660. The medicament container 610 has a proximal end portion 611 and a distal end portion 615. The medicament container 610, which can be constructed of glass, for example, is configured to contain a medicament 617, such as, for example, a dermal filler, a sub-dermal filler, a therapeutic substance for mesotherapy or the like. The medicament container 610 includes a plunger 619 configured to displace the medicament 617 within the medicament container 610. More particularly, when the medical injector 600 is actuated, the plunger 619 is moved within the medicament container 610, in a direction indicated by the arrow HH, thereby causing the medicament 617 to be conveyed into a patient's body.

The needle assembly 620 includes a needle 621 and a hub 624. As shown in FIG. 16, the hub 624 includes a distal end portion 627 and a proximal end portion 625. The distal end portion 627 of the hub 624 defines a shoulder 628 against which a portion of the retainer 640 is disposed. The needle 621 is disposed within the distal end portion 627 of the hub 624. The needle 621 and the hub 624 are collectively coupled to the distal end portion 615 of the medicament container 610 via the coupler 630 such that the needle 621 is in fluid communication with the medicament container 610. The coupler 630 provides a substantially fluid-tight seal between the needle assembly 620 and the medicament container 610 such that the medicament 617 does not leak from the interface between the medicament container 610 and the needle assembly 620 when the medicament 617 is conveyed from the medicament container 610 via the needle 621, as described above.

The adapter 650 includes a proximal end portion 652 and a distal end portion 655, and defines an opening (not shown) therethrough. The proximal end portion 652 of the adapter 650 includes female threads (not shown) configured to receive male threads (not shown) of the actuator 660. Said another way, the adapter 650 is threadedly coupled to the actuator 660. The adapter 650 is disposed about a flanged portion (not shown) of the proximal end portion 611 of the medicament container 610, as described above, such that the medicament container 610 is operatively coupled to the actuator 660 via the adapter 650. In this manner, the actuator 660 and the medicament container 610 are removeably coupled to the adapter 650. Additionally, the distal end portion 655 of the adapter 650 includes female threads (not shown) configured to receive the male threads of the retainer 640, as described herein. In this manner, the adapter 650 is also configured to couple the retainer 640 about the medicament container 610.

The actuator 660 is configured to move the plunger 619 within the medicament container 610 such that the medicament 617 is conveyed to the patient's body via the needle 621. In some embodiments, the actuator 660 can include a source of pressurized fluid that exerts a force on the plunger 619, as described above. In some embodiments, the actuator 660 can actuate the plunger 619 indirectly by acting on a rod (not shown) coupled to the plunger 619 such that the rod moves the plunger 619. In other embodiments, the actuator 660 can be a mechanical device configured to move the plunger.

The retainer 640, which can be constructed from a transparent, translucent and/or opaque material, such as glass, polycarbonate, plexiglass or the like, includes a proximal end portion 641 and a distal end portion 643, and defines a lumen 648 therethrough. The medicament container 610 is disposed within the lumen 648 of the retainer 640 such that the medicament container 610 is substantially enclosed by the retainer 640. In some alternative embodiments, a portion smaller than the entire medicament container 610 is enclosed by the retainer 640. The distal end portion 643 of the retainer 640 defines an opening 647 within which the hub 624 of the needle assembly 620 is disposed. As shown in FIG. 16, the distal end portion 643 of the retainer 640 abuts the shoulder 628 of the hub 624 such that the retainer 640 is secured to the needle assembly 620.

The proximal end portion 641 of the retainer 640 includes male threads 645 configured to threadedly engage the female threads of the adapter 650. Said another way, the retainer 640 is threadedly coupled to the adapter 650. In this manner, the medicament container 610, the retainer 640 and the actuator 660 are coupled together via the adapter 650.

The retainer 640 is configured to limit the movement of the needle assembly 620 relative to the medicament container 610. Additionally, the retainer 640 is configured to limit the movement of the medicament container 610 relative to the adapter 650. Moreover, the retainer 640 is configured to contain the medicament 617 if the medicament container 610 bursts, as described herein.

In use, the retainer 640 is coupled to the medical injector 600 before an injection occurs. First, the medical injector 600 is inserted into the lumen 648 of the retainer 640 such that the medicament container 610 is substantially enclosed by the retainer 640. The needle assembly 620 fits through the opening 647 of the distal end portion 643 of the retainer 640 such that the needle 621 is exposed and the distal end portion 643 of the retainer 640 abuts the shoulder 628 of the hub 624, thereby limiting any further movement. The proximal end portion 641 of the retainer 640 is then threadedly coupled to the adapter 650. More particularly, the threads 645 of the retainer 640 are threaded on to the adapter 650 such that the medicament container 610, the actuator 660 and the retainer 640 are coupled via the adapter 650.

A distal end portion of the needle 621 of the medical injector 600 can be inserted into the patient's body (not shown). The medical injector 600 can then be actuated such that the medicament 617 in the medicament container 610 is conveyed from the medicament container 610 to the patient's body via the needle 621. More specifically, the actuator 660 causes the plunger 619 to move the medicament 617 from the needle 621 in a direction HH, as shown in FIG. 16. In some instances, the pressure of the medicament 617 in the medicament container 610, which can exceed 70 p.s.i., can cause the coupler 630 and/or a portion of the needle assembly 620 to fail such that the needle assembly 620 detaches from the medicament container 610. In other instances, the pressure of the medicament 617 in the medicament container 610 can cause the medicament container 610 and/or the adapter 650 to fail such that the medicament container 610 detaches from the adapter 650. When such detachment occurs, the retainer 640 maintains the position of the needle assembly 620 and/or the medicament container 610. Said another way, the retainer 640 prevents the needle assembly 620 and/or the medicament container 610 from moving relative to the adapter. Additionally, the retainer 640 contains any medicament 617 that leaks from the medicament container 610 such that no medicament 617 is wasted as a result of said detachment.

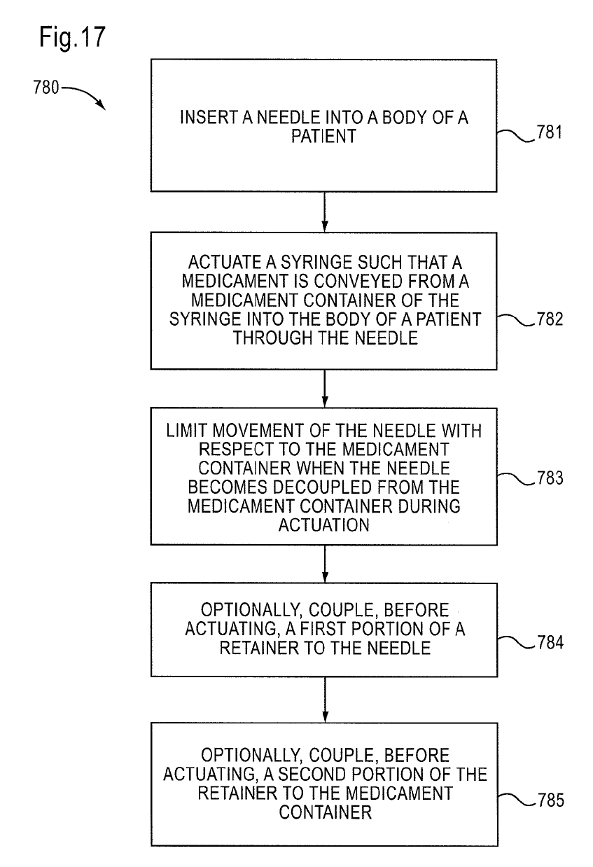

FIG. 17 is a flow chart of a method 780 of using a medical injector according to an embodiment of the invention. The medical injector includes a needle and a syringe. The method includes inserting the needle into a body of a patient, 781. In some embodiments, the inserting includes inserting the needle of the syringe into a skin of the body of the patient to a depth of at least 1 mm. In other embodiments, such as, for example, an injection associated with mesotherapy, the needle can be inserted into the skin to a depth of between 1 mm and 2 mm. In yet other embodiments, such as, for example, an injection associated with intradermal therapy, the needle can be inserted into the skin to a depth of between 2 mm and 5 mm. In yet other embodiments, such as, for example, an injection associated with subdermal therapy, the needle can be inserted into the skin to a depth greater than 5 mm.

The method includes actuating the syringe such that a medicament is conveyed from a medicament container of the syringe into the body of the patient through the needle, 782. The medicament can be, for example, a dermal filler, a sub-dermal filler, a therapeutic substance for mesotherapy or the like. The actuating includes any of the methods described above. In some embodiments, the actuating further includes applying a compressed gas to a proximal end of the medicament container of the syringe and moving a plunger within the medicament container such that a pressure of the medicament within the medicament container is greater than 70 p.s.i. In some embodiments, the actuating includes actuating a machine (e.g., a stepper motor) that actuates the medicament container of the syringe.

The method includes limiting movement of the needle with respect to the medicament container when the needle becomes decoupled from the medicament container during the actuation, 783. The limiting movement can include any of the methods described above. In some embodiments, the limiting movement can be provided by a retainer. For example, the limiting movement can include limiting movement via a retainer such that a distal end of the retainer is coupled to the needle of the syringe and a proximal end of the retainer is coupled to the medicament container of the syringe, as described herein. In some embodiments, the method can further include coupling a first portion of a retainer to the needle of the syringe, before the actuating, 784. Additionally, the method can include coupling a second portion of the retainer to the medicament container of the syringe, before the actuating, such that the limiting movement is performed by the retainer, 785.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Although the needles are shown and described above as generally being hypodermic needles, in other embodiments, a needle can include any suitable injection member, such as a cannula, trocar, or the like, configured to convey a convey a medicament to the patient's body. For example, in some embodiments, a needle can include a relatively large-bore trocar configured to puncture the skin and convey a medicament therein. In some embodiments, for example, a needle can include an 18 gauge or larger trocar. In other embodiments, a needle includes a relatively short delivery member configured to be coupled to a jet injector. Such delivery members can be, for example, relatively small-bore needles configured to make a small incisions. Such delivery members can have a length of less than 5 mm.

Although the retainer 240 is shown and described above as defining an opening 247 within which the hub 224 of the needle assembly 220 is disposed, in other embodiments, the retainer 240 can include coupling portion (not shown in FIGS. 3-6) configured to be wrapped around at least a portion of the needle hub 224. For example, in some embodiments, the distal end portion 243 of the retainer 240 can include a tether configured to be disposed about at least a portion of the needle hub 224. In this manner, the retainer 240 can be coupled to the needle assembly 220 without requiring that a portion of the retainer 240 be disposed about and/or in contact with the needle 221. This arrangement can allow a user to couple the retainer 240 to the needle assembly 220 without potentially touching the needle 221, thus maintaining the sterility of the needle 221.

Although the retainer 340 is shown and described above as including two end portions (341 and 343) each having an adhesive portion (345 and 346), in other embodiments, a retainer can include any suitable number of adhesive portions. For example, in some embodiments, a retainer can include three or more adhesive portions. In other embodiments, a retainer can include a single adhesive portion configured to substantially surround a medicament container

Although the adapter 650 is described above as one piece configured to couple the medicament container 610, the actuator 660 and the retainer 640, in some embodiments, the adapter 650 can include more than one piece configured to couple the medicament container 610, the actuator 660 and the retainer 640 together.

Although FIGS. 10 through 13 show a rigid retainer 540 having a proximal end portion 541 coupled to the adapter 550 via openings 557 and 554, in some embodiments, the proximal end portion 541 of the retainer 540 can include an adhesive portion such that the retainer 540 can be adhesively coupled to the adapter 550.

Although the flexible retainer 240 is shown in FIG. 4 as bending at a location 249 during installation, in some embodiments, the retainer can plastically deform during installation. For example, in some embodiments, the retainer 240 can be made of a metal foil such that the retainer 240 plastically deforms at location 249 when the retainer 240 is moved to the second configuration. Specifically, the portion 249 of the retainer 240 plastically deforms when the retainer is coupled to the needle assembly 220 and/or the medicament container 210. The plastically deformed portion 249 of the retainer 240 can include, for example, a perforated portion configured to allow the retainer 240 to move from a first configuration (see FIG. 3) to a second configuration (see FIG. 4).

Although embodiments described above show a distal end portion of a retainer being coupled to a hub of a needle assembly, in some embodiments, the distal end portion of the retainer can be coupled to the needle. In some embodiments, the distal end portion of the retainer can be coupled to both the needle and the hub of the needle assembly.

Although embodiments above describe a medicament container made of glass, in some embodiments, the medicament container can be made of plastic, metal or any other suitable material. In some embodiments, for example, the medicament container can be made of glass having a polymer coating on the surface of the medicament container. In yet other embodiments, the medicament container can be made of any suitable clear, opaque, translucent and/or rigid material.

Although the medical injectors are shown and described above as including a rigid medicament container, in other embodiments, a medical injector can be any suitable mechanism for conveying a medicament into the body. For example, in some embodiments, a medical injector can include a flexible medicament container of the types shown and described in U.S. Provisional Application Ser. No. 60/993,541, entitled “Controlled Injection Device,” filed Sep. 12, 2007, which is incorporated herein by reference in its entirety. In other embodiments, a medical injector can be a medical injector of the types shown and described in U.S. Provisional Application Ser. No. 60/857,755, entitled “Injection Device,” filed Nov. 8, 2006, U.S. Provisional Application Ser. No. 60/964,066, entitled “Controlled Injection Device,” filed Aug. 8, 2007, and International Patent Application No. PCT/US2007/023226, entitled “Compositions, Devices and Methods for Modifying Soft Tissue,” filed Nov. 1, 2007, each of which is incorporated herein by reference in its entirety. In yet other embodiments, a medical injector can be a medical injector of the types shown and described in U.S. Provisional Application Ser. No. 61/016,223, entitled “Self-Contained Pressurized Injection Device,” filed Dec. 21, 2007 and U.S. patent application Ser. No. 12/114,194, entitled “Apparatus and Methods for Injecting High Viscosity Dermal Fillers,” filed May 2, 2008, each of which is incorporated herein by reference in its entirety.

Although various embodiments have been described as having particular features and/or combinations of components, other embodiments are possible having a combination of any features and/or components from any of embodiments where appropriate. For example, in some embodiments, the medical injectors 100 and 200 in FIGS. 1 through 8 can include an actuator coupled to the proximal end portions 111 and 211 of the medicament containers 110 and 210. In some such embodiments, the actuator is configured to move a plunger within the medicament containers 110 and 210 such that the medicament can be conveyed through the needles 121 and 221. In some embodiments, the actuator further includes a container configured to produce a pressurized gas, as described above.

1. An apparatus comprising:
a medicament container configured to contain a medicament, the medicament container having a distal end portion and a proximal end portion;
a needle assembly including a needle and a hub;
a coupler configured to couple the needle assembly to the distal end portion of the medicament container; and
a retainer configured to be coupled to the medicament container and the needle assembly, the retainer configured to limit movement of the needle assembly when the coupler fails to maintain a position of the needle assembly relative to the distal end of the medicament container.

2. The apparatus of claim 1, wherein the retainer includes a housing substantially enclosing at least a portion of the medicament container.

3. The apparatus of claim 1, wherein the retainer includes a distal end portion and a proximal end portion, the distal end portion of the retainer defining an opening configured to be disposed about the hub, the proximal end portion of the retainer having an adhesive portion configured to be coupled to the medicament container.

4. The apparatus of claim 1, wherein a distal end portion of the retainer includes a coupling portion defining an opening, the opening and a portion of the needle assembly collectively forming an interference fit.

5. The apparatus of claim 1, further comprising:
an actuator coupled to the proximal end portion of the medicament container, the actuator configured to move a plunger within the medicament container such that the medicament can be conveyed through the needle, the actuator including a container configured to produce a pressurized gas.

6. The apparatus of claim 1, wherein a portion of the retainer is configured to be plastically deformed when the retainer is coupled to at least one of the medicament container or the needle assembly.

7. The apparatus of claim 1, wherein the retainer is configured to limit movement of the needle assembly when at least one of a portion of the hub or a portion of the coupler deforms such that the coupler fails to maintain a position of the needle assembly relative to the distal end of the medicament container.

8. An apparatus comprising:
a medicament container configured to contain a medicament, the medicament container including a distal end portion and a proximal end portion;
a needle coupled to the distal end portion of the medicament container;
an adapter coupled to the proximal end portion of the medicament container, the adapter configured to operatively couple the medicament container to an actuator configured to move a plunger within the medicament container such that the medicament can be conveyed through the needle; and
a retainer having a distal end portion configured to be coupled to the needle and a proximal end portion configured to be coupled to the adapter, the retainer configured to limit movement of the needle with respect to the medicament container when the distal end portion of the retainer is coupled to the needle and when the proximal end portion of the retainer is coupled to the adapter.

9. The apparatus of claim 8, wherein the retainer includes a housing substantially enclosing at least a portion of the medicament container.

10. The apparatus of claim 8, wherein the proximal end portion of the retainer is configured to be threadedly coupled to the adapter.

11. The apparatus of claim 8, wherein the proximal end portion of the retainer has an adhesive portion configured to be coupled to the adapter.

12. The apparatus of claim 8, wherein the retainer is configured to limit the movement of the needle with respect to the medicament container when a pressure of the medicament within the medicament container is greater than 70 p.s.i.

13. The apparatus of claim 8, wherein the proximal end portion of the retainer includes a coupling portion configured to be disposed within an opening defined by the adapter.

14. The apparatus of claim 8, wherein the distal end portion of the retainer includes a coupling portion configured to be disposed about a needle hub.

15. The apparatus of claim 8, wherein the distal end portion of the retainer includes a coupling portion defining an opening, the opening and a portion of the needle collectively forming an interference fit.

16. The apparatus of claim 8, wherein the distal end portion of the retainer defines an opening, the opening configured to be disposed about a needle hub.

17. The apparatus of claim 8, wherein a portion of the retainer is configured to be plastically deformed when the proximal end portion of the retainer is coupled to the adapter.

18. The apparatus of claim 8, further comprising:
the actuator, the actuator including a container configured to produce a pressurized gas.

19. The apparatus of claim 8, further comprising:
the actuator, the actuator including a pressure amplifier, the pressure amplifier configured to receive a working fluid at a first pressure and to produce a force on the plunger within the medicament container such that the medicament within the medicament container is at a second pressure greater than the first pressure.

20. The apparatus of claim 8, wherein
the retainer is configured to limit movement of the medicament container with respect to the actuator when the distal end portion of the retainer is coupled to the needle and when the proximal end portion of the retainer is coupled to the adapter.

21. An apparatus comprising:
a medicament container configured to contain a medicament;
a needle assembly including a needle and a hub, the needle assembly coupled to a distal end portion of the medicament container; and
a retainer having a first coupling portion configured to be coupled to a portion of the medicament container, the retainer having a second coupling portion defining an opening, the opening being disposed about the hub, the retainer configured to be plastically deformed when the first coupling portion of the retainer is coupled to the portion of the medicament container.

22. The apparatus of claim 21, wherein the first coupling portion of the retainer includes an adhesive portion configured to couple the first coupling portion of the retainer to an outer surface of the medicament container.

23. The apparatus of claim 21, wherein the opening of the second coupling portion and the hub collectively form an interference fit.

24. The apparatus of claim 21, further comprising:
an actuator coupled to a proximal end portion of the medicament container, the actuator configured to move a plunger within the medicament container such that the medicament is conveyed through the needle when the actuator moves the plunger.

25. The apparatus of claim 21, wherein the retainer is configured to limit the movement of the needle assembly with respect to the medicament container when a pressure of the medicament within the medicament container is greater than 70 p.s.i.

26. The apparatus of claim 21, further comprising:
a coupler configured to couple the needle assembly to the distal end portion of the medicament container.

27-33. (canceled)
